(19) United States
(12) Patent Application Publication
(10) Pub. No.: US 2025/0381363 A1
(43) Pub. Date: Dec. 18, 2025

(54) Method for Short Time Interval-Based Relaxation, Mindfulness, and Stress Reduction Utilizing Active and Passive Intervals Stimulating the Relaxation Response and Vagus Nerve.

(71) Applicant: Mario Green, San Francisco, CA (US)

(72) Inventor: Mario Green, San Francisco, CA (US)

(21) Appl. No.: 18/742,926

(22) Filed: Jun. 13, 2024

(51) Int. Cl. A61M 21/02; A61M 21/00

(52) U.S. Cl. CPC A61M 21/02; A61M 2021/0022

(57) ABSTRACT

The present invention provides a method for short time interval-based relaxation, mindfulness, and stress reduction. By utilizing alternating active and passive intervals, each ranging from 15 to 300 seconds, and incorporating activities that stimulate the vagus nerve, the method promotes effective stress reduction, enhanced mindfulness, and overall relaxation. The Inner Workout app is customizable, adaptable, and designed to fit into various time constraints, ensuring broad applicability and efficiency.

### RELATED APPLICATION

This application claims priority to U.S. Provisional Application No. 63/472,700 filed; 13, June, 2023 entitled, “Method for Short Time Interval-Based Relaxation, Mindfulness, and Stress Reduction Utilizing Active and Passive Intervals Stimulating the Relaxation Response and Vagus Nerve.”

### STATEMENT REGARDING FEDERALLY SPONSORED RESEARCH OR DEVELOPMENT

Not Applicable

### FIELD OF THE INVENTION

The present invention relates to methods for stress reduction, mindfulness, and relaxation. More particularly, it involves a series of Inner Workout Templates organized into short time intervals of mental and physical activities that balance active and passive exercises to stimulate the relaxation response and the vagus nerve.

### BACKGROUND OF THE INVENTION

Traditional methods for stress reduction and mindfulness often require extended periods of time, which can be a barrier for individuals with busy schedules. Moreover, existing methods may not integrate the principles of interval training commonly used in sports and fitness, which have proven effective in physical training regimes. There is a need for a flexible, time-efficient method that leverages interval training principles to promote relaxation, mindfulness, and stress reduction.

### SUMMARY OF THE INVENTION

The invention provides a series of Inner Workout Templates that serve as various embodiments of a method designed to reduce stress, enhance mindfulness, and promote relaxation through short time intervals ranging from 15 to 300 seconds. These templates alternate between active and passive exercises, stimulating the relaxation response and vagus nerve.

The method is structured to offer a variety of activities that engage different parts of the nervous system, combining principles of interval training from sports and fitness with mindfulness and relaxation techniques. The templates cater to different time availabilities and preferences, ranging from 3-minute to 21-minute Inner Workouts.

### DETAILED DESCRIPTION OF THE INVENTION

### 1. Preparatory Phase

### 1. Initial Rating

The user rates their current anxiety and stress level on a scale of 0 (no stress) to 10 (nervous breakdown).
Duration: 15-30 seconds.

### 2. Focusing

The user closes their eyes and focuses on the present moment, creating a judgment-free environment.
Duration: 15-30 seconds.

### 2. Stimulation and Activation Phase

### 1. Trigger Word

The user repeatedly says the word “safe” to stimulate the vagus nerve.
Duration: 15-300 seconds.

### 2. Counting Backwards:

The user looks up and counts backwards from 30.
Duration: 15-300 seconds.

### 3. Laughter

The user laughs hysterically to stimulate the vagus nerve and release endorphins.
Duration: 15-300 seconds.

### 3. Motor Cortex Stimulation and Activation Phase

The user performs various activities for predetermined intervals of 15 to 300 seconds, which include:

### 1. Larynx/Pharynx Stimulation

Activities: Coughing, yawning, and making vocal sounds.

### 2. Tongue Movements

Activities: Moving the tongue in various ways.

### 3. Jaw Movements

Activities: Moving the jaw in different directions.

### 4. Lip Movements

Activities: Moving the lips in various ways.

### 5. Facial Movements

Activities: Moving the face in different ways.

### 6. Eye Movements

Activities: Moving the eyelids and eyeballs in various ways.

### 7. Eyebrow Movements

Activities: Moving the eyebrows in different ways.

### 8. Neck Movements

Activities: Moving the neck in various directions.

### 9. Thumb Movements

Activities: Moving the thumbs in different ways.

### 10. Finger Movements

Activities: Moving each finger in various ways.

### 11. Hand Movements

Activities: Moving the hands in different ways.

### 12. Wrist Movements

Activities: Moving the wrists in various directions.

### 13. Elbow Movements

Activities: Moving the elbows in different ways.

### 14. Shoulder Movements

Activities: Moving the shoulders in various directions.

### 15. Trunk Movements

Activities: Moving the trunk in different ways.

### 16. Hip Movements

Activities: Moving the hips in various directions.

### 17. Knee Movements

Activities: Moving the knees in different ways.

### 18. Ankle Movements

Activities: Moving the ankles in various directions.

### 19. Toe Movements

Activities: Moving the toes in different ways.

### 4. Breathing Techniques

The user practices various breathing techniques for predetermined intervals of 15 to 300 seconds, including:

### 1. Rhythmic Breathing

Inhale and exhale rhythmically and forcefully.

### 2. Deep Breathing

Breathe deeply, slowly, and consciously.

### 3. Non-Rhythmic Breathing

Breathe non-rhythmically and forcefully.

### 4. Focused Inspiration

Breathe rhythmically, focusing on inspiration.

### 5. Focused Expiration

Breathe rhythmically, focusing on expiration.

### 6. Smooth Breathing

Breathe smoothly and rhythmically.

### 7. Fast Breathing

Breathe quickly and smoothly with longer expiration.

### 8. Breath Holding

Hold the breath in or out.

### 5. Mini Meditation and Mindfulness Methodologies

The user practices various mindfulness and meditation techniques for predetermined intervals of 15 to 300 seconds, including:
1. Traditional Mindfulness
2. Traditional Meditation
3. Visualization Meditation
4. Gratitude Meditation
5. Loving Kindness Meditation
6. Body Scan Meditation
7. Focused Attention Meditation
8. Compassion Meditation
9. Breathing and Pranayama
10. Open Monitoring Meditation
11. Effortless Presence Meditation
12. Zen Meditation
13. Vipassana Meditation
14. Mantra Meditation
15. Self-Inquiry Meditation
16. Yoga Meditation
17. Guided Imagery
18. Relaxation Meditation
19. Guided Meditation
20. Multi-Sensory Brainwave Entrainment

### 6. Tactile Stimulation, Drawing, and Writing

The user performs various tactile activities for predetermined intervals of 15 to 300 seconds, including:
1. Tactile Stimulation
2. Tactile Touch and Visualization
3. Tactile Soft Touch
4. Tactile Writing
5. Tactile Drawing

### 7. Relaxation and Stress Reduction Techniques

The user practices various relaxation and stress reduction techniques for predetermined intervals of 15 to 300 seconds, including:
1. Stress Reduction Techniques
2. Heartbeat Coherence
3. Respiratory Coherence
4. Muscle Tensing and Relaxing

### 8. Brainwave, Auditory, or Multi-Sensory Entrainment

The user engages in brainwave, auditory, or multi-sensory entrainment techniques for predetermined intervals of 15 to 300 seconds.

### 9. Vagus Nerve Stimulation Techniques

The user practices various vagus nerve stimulation techniques for predetermined intervals of 15 to 300 seconds, including:
1. Valsalva Maneuver
2. Deep Diaphragmatic Breathing
3. Laughing
4. Smiling
5. Coughing
6. Humming
7. Gargling
8. Vocal Sounds
9. Movements
10. Ear Point Massage
11. Singing
12. Prayer
13. Chanting
14. Carotid Sinus Massage
15. Stomach Muscle Tensing

### Customization and Duration

Each activity or interval (Innerval) ranges from 15 to 300 seconds.
Activities can be customized and combined to create unique sequences (Inner Workouts) lasting 3, 6, 9, 12, or 15 minutes.
Inner Workouts are designed to be flexible and adaptable to individual or group needs.

### 10. Balanced Breathing Innerval

1. Inhale through the nose for 5 seconds and exhale through the nose for 5 seconds.

2. Inhale through the nose for 6 seconds and exhale through the nose for 6 seconds.

3. Inhale through the nose for 4 seconds and exhale through the nose for 4 seconds.

4. Inhale through the nose for 3 seconds and exhale through the nose for 3 seconds.

5. Inhale through the nose for 2 seconds and exhale through the nose for 2 seconds.

6. Inhale through the nose for 1 second and exhale through the nose for 1 second.

### 11. Extended Breathing Innervals

Focusing on extending the exhales:

1. Inhale through the nose for 1 count or second and exhale through the nose for 2 counts or seconds.

2. Inhale through the nose for 1 count or second and exhale through the nose for 3 counts or seconds.

3. Inhale through the nose for 1 count or second and exhale through the nose for 4 counts or seconds.

4. Inhale through the nose for 1 count or second and exhale through the nose for 5 counts or seconds.

5. Inhale through the nose for 1 count or second and exhale through the nose for 6 counts or seconds.

### BRIEF DESCRIPTION OF DRAWINGS

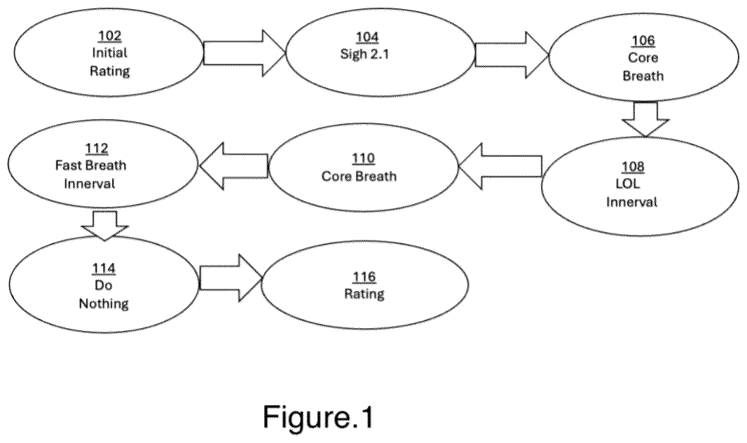

FIG. 1: Flow chart illustrating the Interval Mindfulness: 3 Minute Inner Workout.

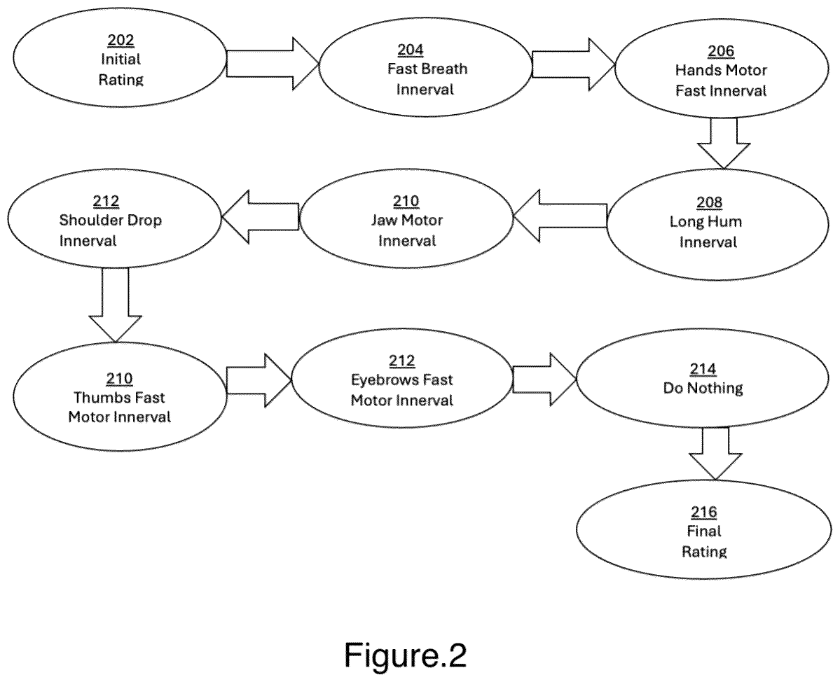

FIG. 2: Flow chart illustrating the Interval Mindfulness: 9 Minute Inner Workout

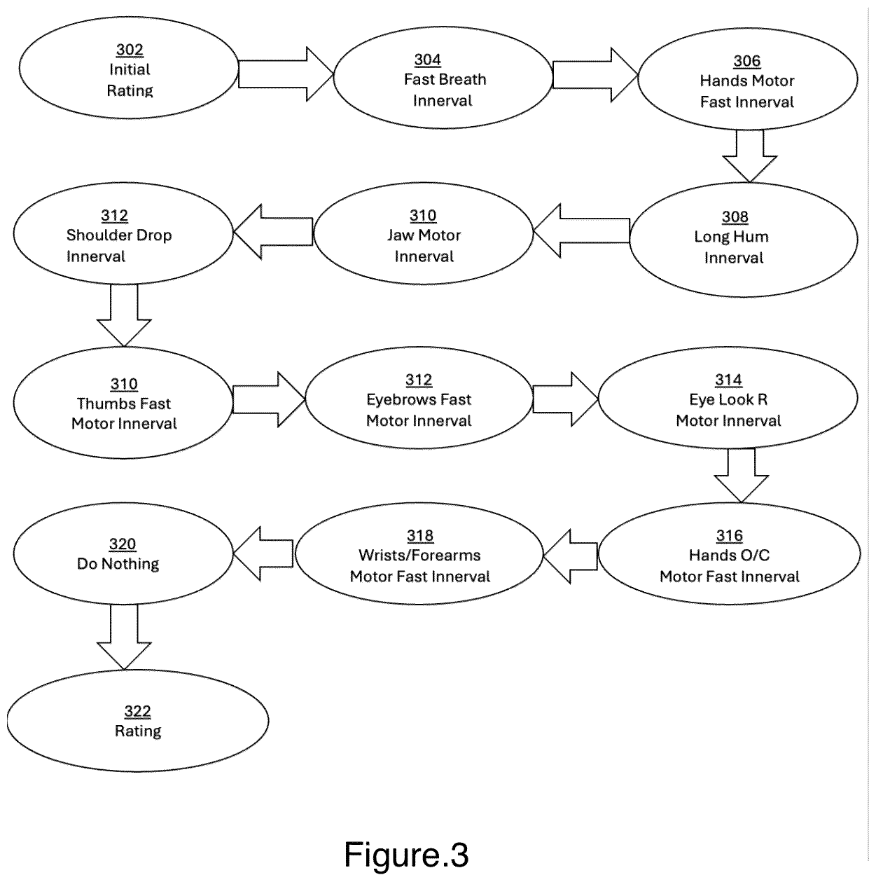

FIG. 3: Flow chart illustrating the Interval Mindfulness: 12 Minute Inner Workout

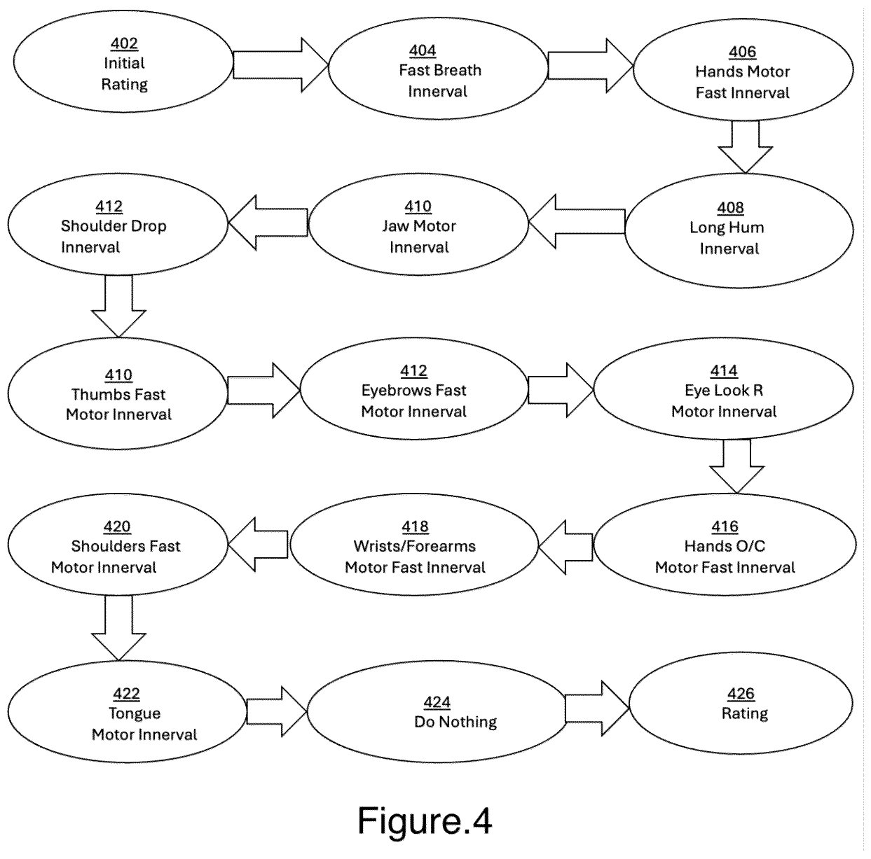

FIG. 4: Flow chart illustrating the Interval Mindfulness: 15 Minute Inner Workout

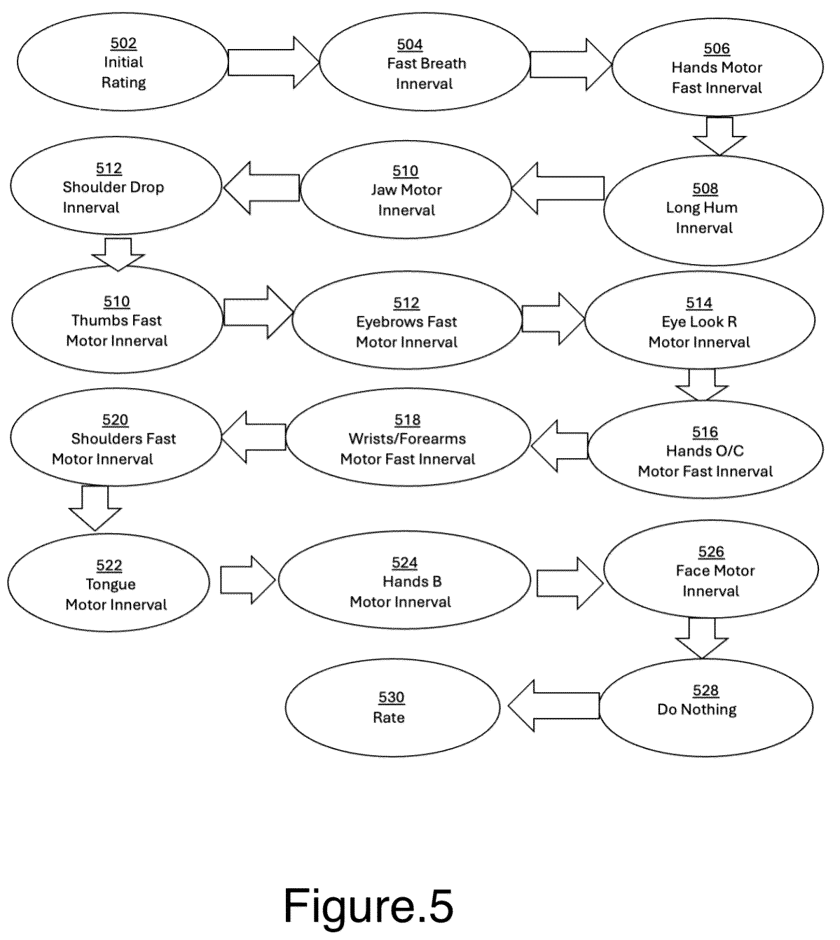

FIG. 5: Flow chart illustrating the Interval Mindfulness: 18 Minute Inner Workout

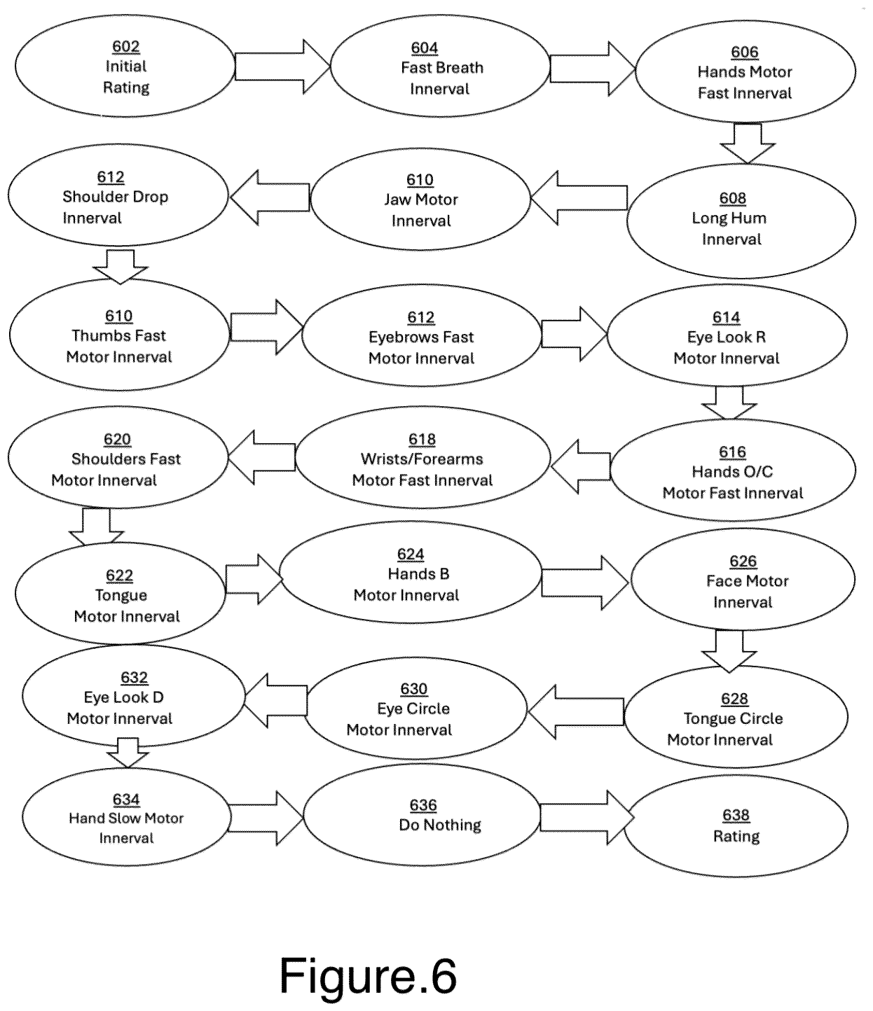

FIG. 6: Flow chart illustrating the Interval Mindfulness: 21 Minute Inner Workout

### DETAILED DESCRIPTION OF DRAWINGS

FIG. 1: Flow chart illustrating the Interval Mindfulness: 3 Minute Inner Workout. This flowchart outlines the sequence of activities in the Inner Workout. Each activity is represented as a node, and the arrows show the flow from one activity to the next. The process starts with 102 the initial rating and proceeds to 104 Sigh 2.1 Innerval the user actively inhales through the nose twice and exhales through the mouth once, producing an audible sigh sound. The emphasis is placed on achieving a high-pitched tone and feeling the vibration at the back of the throat. 106 The user passively inhales, allowing their core to expand outward, and exhales, feeling the core draw inward. 108 The user engages in continuous hysterical laughter for the duration of 30 seconds, faking the laughter if necessary. 110 The user repeats the passive breathing technique of inhaling and allowing the core to expand outward, then exhaling and feeling the core draw inward. 112 The user inhales and exhales through the nose as quickly and equally as possible for 30 seconds or approximately 60 repetitions if a timer is unavailable. 114 The user passively engages in doing nothing, remaining completely still and relaxed. 116 The user re-assesses and rates their current anxiety and stress levels on the same scale of 0 to 10.

FIG. 2: Flow chart illustrating the Interval Mindfulness: 9 Minute Inner Workout. This flowchart outlines the sequence of activities in the Inner Workout. Each activity is represented as a node, and the arrows show the flow from one activity to the next. The process starts with 202 the initial rating before you begin, rate your current anxiety and stress on a scale of 0 (nothing) to 10 (nervous breakdown) and proceeds to 204 Inhale and exhale through your nose equally as fast as you can. Do this for 30 seconds or 60 times if you don't have a timer. 206 Move both of your hands as fast as you can. Do this for 30 seconds or count to 30 if you don't have a timer. 208 Hum using “mmmm” for as long as you can. Continue for 30 seconds or count to 30 if you don't have a timer. 210 Move your jaw from right to left and front to back as fast as you can. Do this for 30 seconds or count to 30 if you don't have a timer. 212 Inhale and lift your shoulders high, then exhale and drop them. Repeat for 30 seconds or 15 times. 214 Move both of your thumbs as fast as you can. Do this for 30 seconds or count to 30 if you don't have a timer. 216 Move your eyebrows up and down as fast as you can. Do this for 30 seconds or count to 30 if you don't have a timer. 218 Remain completely still and relaxed. 220 Rate your current anxiety and stress levels on a scale of 0 (nothing) to 10 (nervous breakdown).

FIG. 3: Flow chart illustrating the Interval Mindfulness: 12 Minute Inner Workout. This flowchart outlines the sequence of activities in the Inner Workout. Each activity is represented as a node, and the arrows show the flow from one activity to the next. The process starts with 302 the initial rating before you begin, rate your current anxiety and stress on a scale of 0 (nothing) to 10 (nervous breakdown) and proceeds to 304 inhale and exhale through your nose equally as fast as you can. Do this for 30 seconds or 60 times if you don't have a timer. 306 Move both of your hands as fast as you can. Do this for 30 seconds or count to 30 if you don't have a timer. 306 Hum using “mmmm” for as long as you can. Continue for 30 seconds or count to 30 if you don't have a timer. 308 Move your jaw from right to left and front to back as fast as you can. Do this for 30 seconds or count to 30 if you don't have a timer. 308 Inhale and lift your shoulders high, then exhale and drop them. Repeat for 30 seconds or 15 times. 310 Move both of your thumbs as fast as you can. Do this for 30 seconds or count to 30 if you don't have a timer. 310 Move your eyebrows up and down as fast as you can. Do this for 30 seconds or count to 30 if you don't have a timer. 312 With your eyes closed, move your eyeballs as far right as possible. Hold for 30 seconds or count to 30. 314 Open and close both hands as fast as you can for 30 seconds. 316 Move both wrists and forearms as fast as you can for 30 second. 318 Remain completely still and relaxed. 320 Rate your current anxiety and stress levels on a scale of 0 (nothing) to 10 (nervous breakdown).

FIG. 4: Flow chart illustrating the Interval Mindfulness: 15 Minute Inner Workout. This flowchart outlines the sequence of activities in the Inner Workout. Each activity is represented as a node, and the arrows show the flow from one activity to the next. The process starts with 402 the initial rating. 404 Inhale and exhale through your nose equally as fast as you can. Do this for 30 seconds or 60 times if you don't have a timer. 406 Move both of your hands as fast as you can. Do this for 30 seconds or count to 30 if you don't have a timer. 408 Hum using “mmmm” for as long as you can. Continue for 30 seconds or count to 30 if you don't have a timer. 408 Move your jaw from right to left and front to back as fast as you can. Do this for 30 seconds or count to 30 if you don't have a timer. 410 Inhale and lift your shoulders high, then exhale and drop them. Repeat for 30 seconds or 15 times. 412 Move both of your thumbs as fast as you can. Do this for 30 seconds or count to 30 if you don't have a timer. 414 Move your eyebrows up and down as fast as you can. Do this for 30 seconds or count to 30 if you don't have a timer. 416 With your eyes closed, move your eyeballs as far right as possible. Hold for 30 seconds or count to 30. 418 Open and close both hands as fast as you can for 30 seconds. 420 Move both wrists and forearms as fast as you can for 30 seconds. 422 Move both of your shoulders as fast as you possibly can for 30 seconds. 424 Move your tongue as fast as you can releasing tension, tightness, and stress any way that you feel that you need to move it. Do this for 30 seconds or count to 30 if you don't have a timer. 426 Remain completely still and relaxed. 428 Rate your current anxiety and stress levels on a scale of 0 (nothing) to 10 (nervous breakdown).

FIG. 5: Flow chart illustrating the Interval Mindfulness: 18 Minute Inner Workout. Flow chart illustrating the Interval Mindfulness: 18 Minute Inner Workout. This flowchart outlines the sequence of activities in the Inner Workout. Each activity is represented as a node, and the arrows show the flow from one activity to the next. The process starts with 502 the initial rating. 504 Inhale and exhale through your nose equally as fast as you can. Do this for 30 seconds or 60 times if you don't have a timer. 506 Move both of your hands as fast as you can. Do this for 30 seconds or count to 30 if you don't have a timer. 506 Hum using “mmmm” for as long as you can. Continue for 30 seconds or count to 30 if you don't have a timer. 508 Move your jaw from right to left and front to back as fast as you can. Do this for 30 seconds or count to 30 if you don't have a timer. 510 Inhale and lift your shoulders high, then exhale and drop them. Repeat for 30 seconds or 15 times. 512 Move both of your thumbs as fast as you can. Do this for 30 seconds or count to 30 if you don't have a timer. 514 Move your eyebrows up and down as fast as you can. Do this for 30 seconds or count to 30 if you don't have a timer. 516 With your eyes closed, move your eyeballs as far right as possible. Hold for 30 seconds or count to 30. 518 Open and close both hands as fast as you can for 30 seconds. 520 Move both wrists and forearms as fast as you can for 30 seconds. 522 Move both of your shoulders as fast as you possibly can for 30 seconds. 524 Move your tongue as fast as you can releasing tension, tightness, and stress any way that you feel that you need to move it. Do this for 30 seconds or count to 30 if you don't have a timer. 526 Move both hands, one at a time, back-and-forth as fast as you possibly can for 30 seconds. 528 Alternate making happy and sad faces. Do this for 30 seconds or 10 face changes if you don't have a timer. 530 Remain completely still and relaxed. 532 Rate your current anxiety and stress levels on a scale of 0 (nothing) to 10 (nervous breakdown).

FIG. 6. Flow chart illustrating the Interval Mindfulness: 21 Minute Inner Workout. This flowchart outlines the sequence of activities in the Inner Workout. Each activity is represented as a node, and the arrows show the flow from one activity to the next. The process starts with 602 the initial rating. 604 Inhale and exhale through your nose equally as fast as you can. Do this for 30 seconds or 60 times if you don't have a timer. 606 Move both of your hands as fast as you can. Do this for 30 seconds or count to 30 if you don't have a timer. 608 Hum using “mmmm” for as long as you can. Continue for 30 seconds or count to 30 if you don't have a timer. 608 Move your jaw from right to left and front to back as fast as you can. Do this for 30 seconds or count to 30 if you don't have a timer. 610 Inhale and lift your shoulders high, then exhale and drop them. Repeat for 30 seconds or 15 times. 612 Move both of your thumbs as fast as you can. Do this for 30 seconds or count to 30 if you don't have a timer. 614 Move your eyebrows up and down as fast as you can. Do this for 30 seconds or count to 30 if you don't have a timer. 616 With your eyes closed, move your eyeballs as far right as possible. Hold for 30 seconds or count to 30. 620 Open and close both hands as fast as you can for 30 seconds. 622 Move both wrists and forearms as fast as you can for 30 seconds. 624 Move both of your shoulders as fast as you possibly can for 30 seconds. 626 Move your tongue as fast as you can releasing tension, tightness, and stress any way that you feel that you need to move it. Do this for 30 seconds or count to 30 if you don't have a timer. 628 Move both hands, one at a time, back-and-forth as fast as you possibly can for 30 seconds. 630 Alternate making happy and sad faces. Do this for 30 seconds or 10 face changes if you don't have a timer. 632 Place the tip of your tongue on the roof of your mouth and draw continuous circles for 30 seconds. 634 Move your eyeballs in a big circle to the right as slowly as possible for 30 seconds. 636 Look down as low as you can with your eyes closed and hold for 30 seconds. 638 Move both of your hands as slow as you can for 30 seconds. 640 Remain completely still and relaxed. 642 Rate your current anxiety and stress levels on a scale of 0 (nothing) to 10 (nervous breakdown).

What is claimed:

1. A method for short time interval-based relaxation, mindfulness, and stress reduction, comprising: a. Conducting an initial rating of the user's stress and anxiety levels; b. Focusing on the present moment with eyes closed; c. Alternating between active and passive intervals, each ranging from 15 to 300 seconds, including: i. Trigger words for vagus nerve stimulation; ii. Backwards counting for brain activation; iii. Laughter for endorphin release; iv. Motor cortex stimulation activities; v. Breathing techniques; vi. Mini meditation and mindfulness methodologies; vii. Tactile stimulation, drawing, and writing; viii. Relaxation and stress reduction techniques; ix. Brainwave, auditory, or multi-sensory entrainment; x. Vagus nerve stimulation techniques. d. Customizing and combining intervals to create Inner Workouts lasting 3, 6, 9, 12, or 15 minutes.

2. The method of claim 1, wherein the initial rating is conducted for a duration of 15-30 seconds.

3. The method of claim 1, wherein the focusing step is conducted for a duration of 15-30 seconds.

4. The method of claim 1, wherein the active intervals include motor cortex stimulation activities involving movements of various body parts, such as the larynx, tongue, jaw, lips, face, eyes, eyebrows, neck, thumbs, fingers, hands, wrists, elbows, shoulders, trunk, hips, knees, ankles, and toes.

5. The method of claim 1, wherein the breathing techniques include rhythmic breathing, deep breathing, non-rhythmic breathing, focused inspiration, focused expiration, smooth breathing, fast breathing, and breath holding.

6. The method of claim 1, wherein the mini meditation and mindfulness methodologies include traditional mindfulness, traditional meditation, visualization meditation, gratitude meditation, loving kindness meditation, body scan meditation, focused attention meditation, compassion meditation, breathing and pranayama, open monitoring meditation, effortless presence meditation, Zen meditation, Vipassana meditation, mantra meditation, self-inquiry meditation, yoga meditation, guided imagery, relaxation meditation, guided meditation, and multi-sensory brainwave entrainment.

7. The method of claim 1, wherein the tactile stimulation activities include tactile stimulation, tactile touch and visualization, tactile soft touch, tactile writing, and tactile drawing.

8. The method of claim 1, wherein the relaxation and stress reduction techniques include various stress reduction methods, heartbeat coherence, respiratory coherence, and muscle tensing and relaxing.

9. The method of claim 1, wherein the vagus nerve stimulation techniques include the Valsalva maneuver, deep diaphragmatic breathing, laughing, smiling, coughing, humming, gargling, vocal sounds, specific movements, ear point massage, singing, prayer, chanting, carotid sinus massage, and stomach muscle tensing.
